(19) United States
(12) Patent Application Publication
(10) Pub. No.: US 2010/0330177 A1
(43) Pub. Date: Dec. 30, 2010

(54) PHARMACEUTICAL COMPOSITIONS OF A COMBINATION OF METFORMIN AND A DIPEPTIDYL PEPTIDASE-IV INHIBITOR

(75) Inventor: Nazaneen Pourkavoos, Ambler, PA (US)

Correspondence Address: MERCK, P O BOX 2000, RAHWAY, NJ, 07065-0907, US

(73) Assignee: Merck Sharp & Dohme Corp.

(21) Appl. No.: 12/864,885

(22) PCT Filed: Jan. 15, 2009
(86) PCT No.: PCT/US09/31087

§ 371 Date: Jul. 28, 2010

(51) Int. Cl. A61K 9/30; A61K 31/4985; A61P 3/10

(52) U.S. Cl. 424/465; 514/249

(57) ABSTRACT

Disclosed are pharmaceutical compositions comprising fixed-dose combinations of an extended-release form of metformin, or a pharmaceutically acceptable salt thereof, coated with an immediate-release form of the DPP-4 inhibitor sitagliptin, or a pharmaceutically acceptable salt thereof.

### BACKGROUND OF THE INVENTION

Type 2 diabetes is a chronic and progressive disease arising from a complex pathophysiology involving the dual endocrine defects of insulin resistance and impaired insulin secretion. The treatment of Type 2 diabetes typically begins with diet and exercise, followed by oral antidiabetic monotherapy. For many patients, these regimens do not sufficiently control glycemia during long-term treatment, leading to a requirement for combination therapy within several years following diagnosis. However, co-prescription of two or more oral antidiabetic drugs may result in treatment regimens that are complex and difficult for many patients to follow. Combining two or more oral antidiabetic agents into a single tablet provides a potential means of delivering combination therapy without adding to the complexity of patients' daily regimens. Such formulations have been well accepted in other disease indications, such as hypertension (HYZAAR™ which is a combination of losartan potassium and hydrochlorothiazide) and cholesterol lowering (VYTORIN™ which is a combination of simvastatin and ezetimibe). The selection of effective and well-tolerated treatments is a key step in the design of a combination tablet. Moreover, it is essential that the components have complementary mechanisms of action and compatible pharmacokinetic profiles. Examples of marketed combination tablets containing two oral antidiabetic agents include Glucovance™ (metformin and glyburide), Avandamet™ (metformin and rosiglitazone), and Metaglip™ (metformin and glipizide).

Metformin represents the only oral antidiabetic agent proven to reduce the total burden of microvascular and macrovascular diabetic complications and to prolong the lives of Type 2 diabetic patients. Furthermore, metformin treatment is often associated with reductions in body weight in overweight patients and with improvements in lipid profiles in dyslipidemic patients. Metformin hydrochloride is marketed in the U.S. and elsewhere as either immediate-release or extended-release formulations with tablet dosage strengths of 500, 750, 850, and 1000 milligrams. Extended-release formulations of metformin have advantages over immediate-release in terms of affording a more uniform maintenance of blood plasma active drug concentrations and providing better patient compliance by reducing the frequency of administration required.

Dipeptidyl peptidase-IV (DPP-4) inhibitors represent a new class of agents that are being developed for the treatment or improvement in glycemic control in patients with Type 2 diabetes. Specific DPP-4 inhibitors either already approved for marketing or under clinical development for the treatment of Type 2 diabetes include sitagliptin, vildagliptin, saxagliptin, melogliptin, alogliptin, denagliptin, carmegliptin, linagliptin, dutogliptin, P93/01 (Prosidion), Roche 0730699, TS021 (Taisho), and E3024 (Eisai). For example, oral administration of sitagliptin, vildagliptin, alogliptin, and saxagliptin to human Type 2 diabetics has been found to reduce fasting glucose and postprandial glucose excursion in association with significantly reduced HbA_{1c} levels. For reviews on the application of DPP-4 inhibitors for the treatment of Type 2 diabetes, reference is made to the following publications: (1) A. H. Stonehouse, et al., “Management of Type 2 diabetes: the role of incretin mimetics, Exp. Opin. Pharmacother., 7: 2095-2105 (2006); (2) B. D. Green, et al., “Inhibition of dipeptidyl peptidase-IV activity as a therapy of Type 2 diabetes,” Exp. Opin. Emerging Drugs, 11: 525-539 (2006); (3) M. M. J. Combettes, “GLP-1 and Type 2 diabetes: physiology and new clinical advances,” Curr. Opin. Pharmacol., 6: 598-605 (2006); and R. K. Campbell, “Rationale for Dipeptidyl Peptidase 4 Inhibitors: A New Class of Oral Agents for the Treatment of Type 2 Diabetes Mellitus,” Ann. Pharmacother., 41: 51-60 (2007).

Sitagliptin phosphate having structural formula I below is the dihydrogenphosphate salt of (2R)-4-oxo-4-[3-(trifluoromethyl)-5,6-dihydro[1,2,4]triazolo[4,3-a]pyrazin-7(8H)-yl]-1-(2,4,5-trifluorophenyl)butan-2-amine.

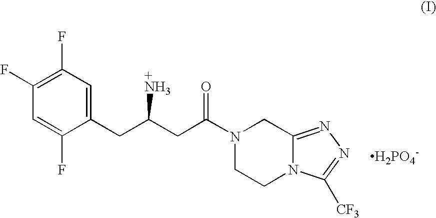

In one embodiment sitagliptin phosphate is in the form of a crystalline monohydrate. Sitagliptin free base and pharmaceutically acceptable salts thereof are disclosed in U.S. Pat. No. 6,699,871, the contents of which are hereby incorporated by reference in their entirety. Crystalline sitagliptin phosphate monohydrate is disclosed in U.S. Pat. No. 7,326,708, the contents of which are hereby incorporated by reference in their entirety. Sitagliptin phosphate has been approved for marketing in several countries, including the U.S., Europe, Canada, and Mexico, for the treatment of Type 2 diabetes and is branded as JANUVIA™ in the U.S. and elsewhere. For reviews, see D. Drucker, et al., “Sitagliptin,” Nature Reviews Drug Discovery, 6: 109-110 (2007); C. F. Deacon, “Dipeptidyl peptidase 4 inhibition with sitagliptin: a new therapy for Type 2 diabetes,” Exp. Opin. Invest. Drugs, 16: 533-545 (2007); K. A. Lyseng-Williamson, “Sitagliptin,” Drugs, 67: 587-597 (2007); and B. Gallwitz, “Sitagliptin: Profile of a Novel DPP-4 Inhibitor for the Treatment of Type 2 Diabetes (Update),” Drugs of Today, 43: 801-814 (2007).

The combination of sitagliptin and metformin provides substantial and additive glycemic improvement in patients with Type 2 diabetes (B. J. Goldstein, et al., “Effect of Initial Combination Therapy with Sitagliptin, a DPP-4 Inhibitor, and Metformin on Glycemic Control in Patients with Type 2 Diabetes,” Diabetes Care, 30: 1979-1987 (2007) and B. Gallwitz, “Sitagliptin with Metformin: Profile of a combination for the treatment of Type 2 diabetes,” Drugs of Today, 43: 681-689 (2007). A fixed-dose combination of immediate-release of both metformin and sitagliptin has been approved for marketing in several countries, including U.S.<Europe, and Mexico, for adult patients with Type 2 diabetes who are not adequately controlled on metformin or sitagliptin alone or in patients already being treated with the combination of sitagliptin and metformin. The combination is branded as JANUMET™ in the U.S. JANUMET™ tablets contain 50 mg sitagliptin and either 500 or 1000 mg metformin. Pharmaceutical compositions comprising fixed-dose combinations of immediate-release sitagliptin and immediate-release metformin are disclosed in PCT international patent application WO 2007/078726 which published on Jul. 12, 2007.

Extended-release formulations of metformin are disclosed in U.S. Pat. No. 6,340,475; U.S. Pat. No. 6,635,280; U.S. Pat. No. 6,866,866; U.S. Pat. No. 6,475,521; and U.S. Pat. No. 6,660,300. Pharmaceutical formulations containing extended-release metformin and a thiazolidinedione antihyperglycemic agent are described in WO 2004/026241 (1 Apr. 2004) and WO 2006/107528 (12 Oct. 2006). Pharmaceutical compositions comprising a DPP-4 inhibitor and a slow-release form of metformin are disclosed in US 2007/0172525 (26 Jul. 2007) and US 2008/0064701 (13 Mar. 2008). Stable pharmaceutical compositions of an immediate-release form of the antihyperglycemic sulfonylurea glimepiride and extended-release metformin are disclosed in US 2007/0264331 (15 Nov. 2007).

The present invention provides for pharmaceutical compositions of a fixed-dose of an extended-release form of metformin coated with an immediate release form of sitagliptin which are prepared by wet or dry processing methods. In one embodiment the pharmaceutical compositions of the present invention are in the dosage form of a tablet, and, in particular, a film-coated tablet.

The present invention also provides processes to prepare pharmaceutical compositions of a fixed-dose combination of sitagliptin and metformin by wet or dry processing methods. The wet processing methods include wet granulation.

Another aspect of the present invention provides methods for the treatment of Type 2 diabetes by administering to a host in need of such treatment a therapeutically effective amount of a pharmaceutical composition of the present invention.

These and other aspects of the invention will become readily apparent from the detailed description which follows.

### SUMMARY OF THE INVENTION

The present invention is directed to novel pharmaceutical compositions comprising an extended-release form of metformin, or a pharmaceutically acceptable salt thereof, coated with an immediate-release form of the DPP-4 inhibitor sitagliptin, or a pharmaceutically acceptable salt thereof, processes for preparing such compositions, and methods of treating Type 2 diabetes with such compositions. In particular, the invention is directed to pharmaceutical compositions comprising an extended-release form of metformin hydrochloride coated with an immediate-release form of sitagliptin phosphate.

### BRIEF DESCRIPTION OF THE FIGURES

FIG. 1 is a graph showing in vitro dissolution profiles comparing immediate-release (IR) tablets containing 500 milligrams metformin hydrochloride with extended-release (matrix) tablet cores containing 500, 850, or 1000 milligrams metformin hydrochloride.

FIG. 2 is a graph showing in vitro dissolution profiles for sitagliptin phosphate from the drug film layer in a pharmaceutical composition of the present invention compared to sitagliptin phosphate in JANUMET™ which is a marketed fixed-dose combination of immediate-release metformin hydrochloride and immediate-release sitagliptin phosphate.

### DETAILED DESCRIPTION OF THE INVENTION

One aspect of the present invention is directed to pharmaceutical compositions comprising a fixed-dose combination of an extended-release form of metformin, or a pharmaceutically acceptable salt thereof, coated with an immediate-release form of the DPP-4 inhibitor sitagliptin, or a pharmaceutically acceptable salt thereof. The pharmaceutical compositions are formulated into dosage forms suitable for the simultaneous medicinal administration of the two antihyperglycemic agents. A particular solid dosage form relates to tablets comprising a fixed-dose combination of an extended-release form of metformin hydrochloride coated with an immediate-release form of sitagliptin phosphate.

A preferred pharmaceutically acceptable salt of sitagliptin is the dihydrogenphosphate salt of structural formula I above (sitagliptin phosphate). A preferred form of the dihydrogenphosphate salt is the crystalline monohydrate disclosed in U.S. Pat. No. 7,326,708, the contents of which are hereby incorporated by reference in their entirety.

The preparation of sitagliptin, and pharmaceutically acceptable salts thereof, is disclosed in U.S. Pat. No. 6,699,871, the contents of which are herein incorporated by reference in their entirety. The preparation of sitagliptin phosphate monohydrate is disclosed in U.S. Pat. No. 7,326,708, the contents of which are hereby incorporated by reference in their entirety.

The unit dosage strength of sitagliptin free base anhydrate (active moiety) for inclusion into the fixed-dose combination pharmaceutical compositions of the present invention is 25, 50, and 100 milligrams. An equivalent amount of sitagliptin phosphate monohydrate to the sitagliptin free base anhydrate is used in the pharmaceutical compositions, namely, 32.125, 64.25 and 128.5 milligrams, respectively.

The unit dosage strength of the metformin hydrochloride for incorporation into the fixed-dose combination of the present invention is 500, 750, 850, and 1000 milligrams. These unit dosage strengths of metformin hydrochloride represent the dosage strengths approved in the U.S. for marketing to treat Type 2 diabetes.

Specific embodiments of dosage strengths for sitagliptin and metformin hydrochloride in the fixed-dose combinations of the present invention are the following:
(1) 25 milligrams of sitagliptin (equivalent to 32.125 milligrams of sitagliptin phosphate monohydrate) and 250 milligrams metformin hydrochloride;
(2) 25 milligrams of sitagliptin (equivalent to 32.125 milligrams of sitagliptin phosphate monohydrate) and 500 milligrams metformin hydrochloride;
(3) 25 milligrams of sitagliptin (equivalent to 32.125 milligrams of sitagliptin phosphate monohydrate) and 750 milligrams metformin hydrochloride;
(4) 25 milligrams of sitagliptin (equivalent to 32.125 milligrams of sitagliptin phosphate monohydrate) and 850 milligrams metformin hydrochloride;
(5) 25 milligrams of sitagliptin (equivalent to 32.125 milligrams of sitagliptin phosphate monohydrate) and 100 milligrams metformin hydrochloride;
(6) 50 milligrams of sitagliptin (equivalent to 64.25 milligrams of sitagliptin phosphate monohydrate) and 500 milligrams metformin hydrochloride;
(7) 50 milligrams of sitagliptin (equivalent to 64.25 milligrams of sitagliptin phosphate monohydrate) and 750 milligrams metformin hydrochloride;
(8) 50 milligrams of sitagliptin (equivalent to 64.25 milligrams of sitagliptin phosphate monohydrate) and 850 milligrams metformin hydrochloride;
(9) 50 milligrams of sitagliptin (equivalent to 64.25 milligrams of sitagliptin phosphate monohydrate) and 1000 milligrams metformin hydrochloride;
(10) 100 milligrams of sitagliptin (equivalent to 128.5 milligrams of sitagliptin phosphate monohydrate) and 500 milligrams metformin hydrochloride;
(11) 100 milligrams of sitagliptin (equivalent to 128.5 milligrams of sitagliptin phosphate monohydrate) and 750 milligrams metformin hydrochloride;
(12) 100 milligrams of sitagliptin (equivalent to 128.5 milligrams of sitagliptin phosphate monohydrate) and 850 milligrams metformin hydrochloride; and
(13) 100 milligrams of sitagliptin (equivalent to 128.5 milligrams of sitagliptin phosphate monohydrate) and 1000 milligrams metformin hydrochloride.

In a particular aspect of the present invention, the pharmaceutical compositions of the present invention comprise an inner core matrix formulation of metformin hydrochloride containing an extended release material. The matrix formulation is compressed into a tablet form. In an embodiment of this aspect of the invention, the extended release material comprises hydroxypropylmethylcellulose (HPMC) having an apparent viscosity grade of at least 10,000 cP when present in a 2% solution in water at 20° C. In a class of this embodiment, the HPMC has an apparent viscosity grade of at least 80,000 cP when present in a 2% solution in water at 20° C. In a subclass of this class, the HPMC has an apparent viscosity grade of about 80,000 cP to about 120,000 cP (nominal value 100,000 cP) when present in a 2% solution in water at 20° C. In another embodiment, the drug loading of metformin hydrochloride is in the range of about 50% to about 70%.

The metformin matrix tablets are prepared by wet or dry processing methods. In one embodiment the metformin matrix tablets are prepared by wet processing methods. In a class of this embodiment the metformin matrix tablets are prepared by wet granulation methods. With wet granulation either high-shear granulation or fluid-bed granulation may be used.

In the high-shear wet granulation process, metformin hydrochloride is first blended with a suitable binding agent using water or an aqueous ethanol mixture as the granulating solvent. In one embodiment the high-shear granulation process uses a tip speed of 3.58 msec with a granulation fluid level of between 3 and 8%. The resulting granules are next dried and sized to produce a mean particle size range of about 500 to about 800 microns. Compacts produced from the resulting granules exhibit a tensile strength of about 2 to about 3 megapascals [MPa] over a compaction pressure range of about 200 to 400 MPa. Embodiments of suitable binding agents include hydroxypropylcellulose (HPC), hydroxypropylmethyl cellulose (HPMC), hydroxyethyl cellulose, starch 1500, polyvinylpyrrolidone (povidone), and co-povidone. A preferred binding agent is polyvinylpyrrolidone (povidone).

The sized metformin granulation is subsequently blended with an extragranular excipient which consists of a high viscosity HPMC as defined above and optionally including a suitable glidant and/or a suitable lubricant to afford a final metformin drug loading of about 50% to about 70%. The tensile strength of the final blend formulation is about 2.0 MPa to about 2.5 MPa over a range of about 200 MPa to about 400 MPa compaction pressure. The final blend is compressed on a rotary press at a compression force of about 30 kiloNewtons (kN) using modified capsule-shaped tooling resulting in a tablet hardness (breaking force) of about 30-35 kiloponds (kp).

Examples of lubricants include magnesium stearate, calcium stearate, stearic acid, sodium stearyl fumarate, hydrogenated castor oil, and mixtures thereof. A preferred lubricant is magnesium stearate or sodium stearyl fumarate or a mixture thereof. Examples of glidants include colloidal silicon dioxide, calcium phosphate tribasic, magnesium silicate, and talc. In one embodiment the glidant is colloidal silicon dioxide and the lubricant is sodium stearyl fumarate.

The composition of a representative metformin core tablet is provided in Table 1.

TABLE 1
Metformin Core Tablet Composition

| | | Final 70% |
|---|---|---|
| | | (w/w) drug |
| Component | Granulation | loading |
| Metformin HCl | 93.0% | 70.0 |
| PVP K 29/32 | 7.0% | 5.27 |
| Intragranular Weight | 100.0% | |
| Methocel ™ K100M* | | 22.23 |
| Colloidal silicon | | 0.50 |
| dioxide | | |
| Sodium stearyl | | 2.0 |
| fumarate | | |
| Total | | 100% |

*A grade of HPMC having an apparent viscosity of 80,000 to 120,000 cP (nominal value 100,000) (2% in water at 20° C.).

In a second aspect of the present invention, the extended-release metformin core tablet is coated with an aqueous suspension of a sitagliptin salt until a final dried solid weight gain corresponding to 25 mg, 50 mg, or 100 mg of sitagliptin is obtained.

The sitagliptin coating suspension is designed to produce a stable solid solution in an immediate-release polymer film so that the drug is substantially present as an amorphous form to allow rapid dissolution and absorption of sitagliptin to take place following ingestion of the dosage form. Embodiments of the film-forming polymer are hydroxypropylmethylcellulose (HPMC), hydroxypropylcellulose (HPC), sodium carboxymethylcellulose, polyvinylpyrrolidone (PVP), and polyvinylalcohol/PEG 3350. A particular form of HPMC for use as a film-forming polymer is HPMC 2910. The coating suspension also optionally contains one or more excipients selected from the group consisting of a plasticizer, such as polyethylene glycol grades 400 to 3350 and triethyl citrate; a dispersing agent, such as hydrated aluminum silicate (Kaolin); a colorant; and an antioxidant to prevent oxidative degradation. The antioxidant is selected from the group consisting of α-tocopherol, γ-tocopherol, δ-tocopherol, extracts of natural origin rich in tocopherol, L-ascorbic acid and its sodium or calcium salts, ascorbyl palmitate, propyl gallate, octyl gallate, dodecyl gallate, butylated hydroxytoluene (BHT), and butylated hydroxyanisole (BHA). In one embodiment, the antioxidant is propyl gallate.

The sitagliptin coating suspension is prepared to a total solids concentration of about 12% to about 17% w/w. The sitagliptin coating suspension is applied to the metformin matrix tablet and the amount of solids deposited in the active pharmaceutical ingredient (“API”) film layer is controlled to achieve the desired sitagliptin dose. The 50 mg sitagliptin phosphate film potency represents one-half the weight gain of the 100 mg potency.

The composition of a representative sitagliptin film coating suspension is provided in Table 2.

TABLE 2
Sitagliptin Aqueous Film Coating Compositions

| | Solid Concentration | Solid Concentration |
|---|---|---|
| Ingredient | at about 12% (w/w) | at about 17% (w/w) |
| Sitagliptin phosphate | 6.0 | 12.0 |
| monohydrate | | |
| Opadry I Clear | | 5.0 |
| HPMC 2910 (6 Cp) | 3.75 | |
| PEG 3350 NF | 0.75 | |
| Kaolin (Compendial) | 1.5 | |
| Propyl gallate | 0.0637 | 0.0637 |
| FD& C blue Lake dye | 0.10 | |
| Water | 87.84 | 82.936 |
| To Make | 100 | 100 |

The film-coating operation is carried out in a conventional perforated vented pan with baffles and is conducted at a controlled exhaust temperature range of about 40° C. to about 44° C. The spray rate and air flow through the coating pan is adjusted to produce a uniform coating and coverage of the entire width of the tablet bed. The amount of the coating suspension applied is controlled by percent weight gain of tablet cores and typically ranges from about 19 to about 22%. This range resulted in sitagliptin drug assay close to the desired 25 mg, 50 mg, or 100 mg with a standard deviation of about 2-4% for content uniformity assay of sitagliptin. The duration of the coating step is about 4-7 hours.

The final pharmaceutical compositions of the present invention are tablets. Such tablets may be further film-coated such as with a mixture of hydroxypropylcellulose and hydroxypropylmethylcellulose containing titanium dioxide and/or other coloring agents, such as iron oxides, dyes, and lakes; a mixture of polyvinyl alcohol (PVA) and polyethylene glycol (PEG) containing titanium dioxide and/or other coloring agents, such as iron oxides, dyes, and lakes; or any other suitable immediate-release film-coating agent(s). The coat provides taste masking and additional stability to the final tablet. A commercial film-coat is Opadry® which is a formulated powder blend provided by Colorcon.

The pharmaceutical tablet compositions of the present invention may also contain one or more additional formulation ingredients selected from a wide variety of excipients known in the pharmaceutical formulation art. According to the desired properties of the pharmaceutical composition, any number of ingredients may be selected, alone or in combination, based upon their known uses in preparing tablet compositions. Such ingredients include, but are not limited to, diluents, compression aids, glidants, disintegrants, lubricants, flavors, flavor enhancers, sweeteners, and preservatives.

The term “tablet” as used herein is intended to encompass compressed pharmaceutical dosage formulations of all shapes and sizes, whether coated or uncoated.

In one embodiment the metformin matrix tablets are prepared by wet granulation (high shear and/or fluid bed). Granulation is a process in which binding agent is added either through the granulating solution or through dry powder addition to a granulator bowl to form granules. The steps involved in the wet granulation method comprise the following:
(1) the active pharmaceutical ingredient metformin hydrochloride is added to the granulator bowl;
(2) optional disintegrants are added to step 1;
(3) for high-shear granulation, the binding agent (such as polyvinylpyrrolidone or hydroxypropylcellulose) is added dry to the granulator bowl and dry mixed for a short period followed by the addition of water with or without a surfactant (such as sodium lauryl sulfate). For fluid bed granulation, the metformin hydrochloride is added to the granulator bowl and the granulating solution comprised of binding agent with or without surfactant in water is added upon fluidization;
(4) granules prepared by high-shear granulation are tray-dried in an oven or dried in a fluid bed dryer. For granules prepared by fluid-bed granulation, granules are dried in a fluid bed dryer;
(5) dried granules are resized in a suitable mill;
(6) hydroxypropylmethylcellulose with an apparent viscosity of at least 10,000 cP to about 800,000 cP is blended with dried sized granules in a suitable blender;
(7) optional diluents (such as microcrystalline cellulose and dibasic calcium phosphate dihydrate) are blended with dried and sized granules in a suitable blender;
(8) lubricants or glidants (such as magnesium stearate and sodium stearyl fumarate) are added to the blend from step 7 in a suitable blender; and
(9) the lubricated granule mixture from step 8 is compressed into the desired tablet image.

The present invention also provides methods for treating Type 2 diabetes by orally administering to a host in need of such treatment a therapeutically effective amount of one of the fixed-dose combination pharmaceutical compositions of the present invention. In one embodiment the host in need of such treatment is a human. In another embodiment the pharmaceutical composition is in the dosage form of a tablet. The pharmaceutical compositions comprising the fixed-dose combination may be administered once-daily (QD), twice-daily (BID), thrice-daily (TID), or four-times daily.

The following examples further describe and demonstrate embodiments within the scope of the present invention. The examples are given solely for the purpose of illustration and are not intended to be construed as limitations of the present invention as many variations thereof are possible without departing from the spirit and scope of the invention.

### Example 1

### Fixed-Dose Combination of 50 or 100 Milligrams of Sitagliptin and 1000 Milligrams of Metformin Hydrochloride Using 12% Total Sitagliptin Phosphate Coating Suspension

| | 100/1000 | 100/1000% | 50/1000 | 50/1000% |
|---|---|---|---|---|
| Ingredient | mg/ tablet | w/w | mg/ tablet | w/w |
| 1. Tablet Core | | | | |
| Metformin HCl | 1000 | 70 | 1000 | 70 |
| PVP K29/32 | 75.29 | 5.27 | 75.29 | 5.27 |
| Methocel K100M | 317.57 | 22.23 | 317.57 | 22.23 |
| Silicon Dioxide | 7.14 | 0.5 | 7.14 | 0.5 |
| Sodium stearyl | 28.57 | 2.0 | 28.57 | 2.0 |
| fumarate | | | | |
| 2. Sitagliptin Coating | | | | |
| Sitagliptin phosphate | 128.52* | 8.997 | 64.26** | 4.50 |
| monohydrate | | | | |
| Propyl gallate | 1.36 | 0.095 | 0.68 | 0.048 |
| HPMC/PEG/ | 130.66 | 9.15 | 65.33 | 32.67 |
| Kaolin/dye | | | | |
| Total Sitagliptin Coat | 260.55 | 18.24% | 130.27 | 9.12 |
| Total Coated Tablet | 1689.12 | 118.245 | 1558.85 | 109.12 |

*Equivalent to 100 mg of sitagliptin free base anhydrate.
**Equivalent to 50 mg of sitagliptin free base anhydrate.

### Steps in the Preparation of Example 1:

(1) metformin hydrochloride was delumped by passing it through a suitable mill;
(2) the delumped metformin and PVP dry binder powder were transferred into a granulator bowl of a high-shear granulator and granulated with water at a level of 3 to 8% of total dry powder batch size until granules were formed;
(3) the granules were dried in an oven at 50° C. to a moisture content of less than 2%;
(4) the dried granules were sized in a suitable mill to obtain a mean granule particle size of about 500-800 microns;
(5) the dried and sized granules were blended with Methocel K100M and pre-screened (mesh #60) silicon dioxide;
(6) the pre-screened (mesh #60) sodium stearyl fumarate and blend from step 5 were blended in a suitable blender to produce the final blend;
(7) the final blend from step 6 was compressed in a rotary tablet press at a main compression force of about 30 kN to produce tablets at the target weight range and hardness;
(8) the sitagliptin phosphate coating suspension was prepared by mixing all the excipients (except Kaolin) and sitagliptin phosphate in the required amount of purified water using a suitable homogenizer until the solids were dissolved;
(9) the pre-screened (mesh #60) Kaolin powder was added to the sitagliptin phosphate coating suspension and mixed with a suitable mixer and blade until the powder was uniformly dispersed in the coating suspension;
(10) the compressed tablet cores from step 7 were loaded into a suitable perforated side-vented coating pan with baffles fitted with single or multiple spray guns to produce a spray fan to cover the entire width of the tablet bed;
(11) the tablet bed was warmed in the rotating coating pan until an exhaust temperature of 40-44° C. was reached at an inlet air flow of about 270-350 cubic feet/min (CFM);
(12) the average weight of warmed uncoated tablet was determined as the initial starting weight;
(13) the sitagliptin phosphate coating suspension was sprayed onto the tablet bed at a suitable spray rate and atomization pressure;
(14) spraying with the sitagliptin phosphate coating suspension was continued while monitoring the tablet weight until the required weight gain was obtained;
(15) an approximate dried coat weight of 130 mg equivalent to 50 mg sitagliptin (as free base) or 260 mg equivalent to 100 mg of sitagliptin (as free base) was deposited over the tablet cores;
(16) spraying was stopped, and the tablets were dried and discharged from the coating pan.

### Example 2

### Fixed-Dose Combination of 50 or 100 Milligrams of Sitagliptin and 1000 Milligrams of Metformin Hydrochloride Using 17% Total Sitagliptin Phosphate Coating Suspension

| | 100/1000 | 100/1000% | 50/1000 | 50/1000% |
|---|---|---|---|---|
| Ingredient | mg/tablet | w/w | mg/tablet | w/w |
| 1. Tablet Core | | | | |
| Metformin HCl | 1000 | 70 | 1000 | 70 |
| PVP K29/32 | 75.29 | 5.27 | 75.29 | 5.27 |
| Methocel K100M | 317.57 | 22.23 | 317.57 | 22.23 |
| Silicon Dioxide | 7.14 | 0.5 | 7.14 | 0.5 |
| Sodium stearyl | 28.57 | 2.0 | 28.57 | 2.0 |
| fumarate | | | | |
| Total Tablet Cores | 1428.57 | 100 | 1428.57 | 100 |
| 2. Sitagliptin Coating | | | | |
| Sitagliptin phosphate | 128.52* | 9.0 | 64.26** | 4.50 |
| monohydrate | | | | |
| Propyl gallate | 0.68 | 0.048 | 0.34 | 0.024 |
| Opadry I Clear | 53.55 | 3.75 | 26.78 | 1.87 |
| Total Sitagliptin Coat | 182.75 | 12.79 | 91.38 | 6.4 |
| Total Coated Tablet | 1611.28 | 112.79% | 1519.99 | 106.4 |

*Equivalent to 100 mg of sitagliptin free base anhydrate.
**Equivalent to 50 mg of sitagliptin free base anhydrate.

### Steps in Preparation of Example 2:

(1) metformin hydrochloride was delumped by passing it through a suitable mill;
(2) the delumped metformin and PVP dry binder powder were transferred into a granulator bowl of a high-shear granulator and granulated with water at a level of 3 to 8% of total dry powder batch size until granules were formed;
(3) the granules were dried in an oven at 50° C. to a moisture content of less than 2%;
(4) the dried granules were sized in a suitable mill to obtain a mean granule particle size of about 500-800 microns;
(5) the dried and sized granules were blended with Methocel K100M and pre-screened (mesh #60) silicon dioxide;
(6) the pre-screened (mesh #60) sodium stearyl fumarate and blend from step 5 were blended to produce the final blend;
(7) the final blend from step 6 was compressed in a rotary tablet press at a main compression force of about 30 kN to produce tablets at the target weight range and hardness;
(8) the sitagliptin phosphate coating suspension was prepared by mixing all the excipients and sitagliptin phosphate in the required amount of purified water using a suitable homogenizer until the solids were uniformly dispersed in the coating suspension;
(9) the compressed tablet cores from step 7 were loaded into a suitable perforated side-vented coating pan with baffles fitted with single or multiple spray guns to produce a spray fan to cover the entire width of the tablet bed;
(10) the tablet bed was warmed in the rotating coating pan until an exhaust temperature of 40-44° C. was reached at an inlet air flow of about 270-350 CFM;
(11) the average weight of warmed uncoated tablet was determined as the initial starting weight;
(12) the sitagliptin phosphate coating suspension was sprayed onto the tablet bed at a suitable spray rate and atomization pressure;
(13) spraying with the sitagliptin phosphate coating suspension was continued while monitoring the tablet weight until the required weight gain was obtained;
(14) an approximate dried coat weight of 91 mg equivalent to 50 mg sitagliptin (as free base) or 182 mg equivalent to 100 mg of sitagliptin (as free base) was deposited over the tablet cores;
(15) spraying was stopped, and the tablets were dried and discharged from the coating pan.

### Example 3

### Fixed-Dose Combination of 50 or 100 Milligrams of Sitagliptin and 1000 Milligrams of Metformin Hydrochloride Using 12% Total Sitagliptin Phosphate Coating Suspension and a 10% Opadry I™ White Suspension

| | 100/1000 | 100/1000% | 50/1000 | 50/1000% |
|---|---|---|---|---|
| Ingredient | mg/tablet | w/w | mg/tablet | w/w |
| 1. Tablet Core | | | | |
| Metformin HCl | 1000 | 70 | 1000 | 70 |
| PVP K29/32 | 75.29 | 5.27 | 75.29 | 5.27 |
| Methocel K100M | 317.57 | 22.23 | 317.57 | 22.23 |
| Silicon Dioxide | 7.14 | 0.5 | 7.14 | 0.5 |
| Sodium stearyl | 28.57 | 2.0 | 28.57 | 2.0 |
| fumarate | | | | |
| Total Tablet Cores | 1428.57 | 100 | 1428.57 | 100 |
| 2. Sitagliptin Coating | | | | |
| Sitagliptin phosphate | 128.52* | 8.997 | 64.26** | 4.50 |
| monohydrate | | | | |
| Propyl gallate | 1.36 | 0.095 | 0.68 | 0.048 |
| HPMC 2910 | 80.33 | 5.623 | 40.165 | 2.81 |
| PEG 3350 | 16.07 | 1.125 | 8.035 | 0.562 |
| Kaolin | 32.13 | 2.249 | 16.07 | 1.125 |
| Total Sitagliptin Coat | 258.41 | 18.09% | 129.21 | 9.05 |
| Total Coated Tablet | 1686.98 | 118.09 | 1557.78 | 109.05 |
| 3. Opadry I White | 33.74 | 2 | 31.15 | 2 |
| Overcoat | | | | |
| Total overcoated | 1720.72 | 120.09 | 1588.93 | 111.05 |
| Tablet | | | | |

*Equivalent to 100 mg of sitagliptin free base anhydrate.
**Equivalent to 50 mg of sitagliptin free base anhydrate.

### Steps in Preparation of Example 3:

(1) metformin hydrochloride was delumped by passing it through a suitable mill;
(2) the delumped metformin and PVP dry binder powder were transferred into a granulator bowl of a high-shear granulator and granulated with water at a level of 3 to 8% of total dry powder batch size until granules were formed;
(3) the granules were dried in an oven at 50° C. to a moisture content of less than 2%;
(4) the dried granules were sized in a suitable mill to obtain a mean granule particle size of about 500-800 microns;
(5) the dried and sized granules were blended with Methocel K100M and pre-screened (mesh #60) silicon dioxide;
(6) the pre-screened (mesh #60) sodium stearyl fumarate and blend from step 5 were blended in a suitable blender to produce the final blend;
(7) the final blend from step 6 was compressed in a rotary tablet press at a main compression force of about 30 kN to produce tablets at the target weight range and hardness;
(8) the sitagliptin phosphate coating suspension was prepared by mixing all the excipients (except Kaolin) and sitagliptin phosphate in the required amount of purified water using a suitable homogenizer until the solids were dissolved;
(9) the pre-screened (mesh #60) Kaolin was added to the sitagliptin phosphate coating suspension and mixed with a suitable mixer and blade until the powder was uniformly dispersed in the coating suspension;
(10) the compressed tablet cores from step 7 were loaded into a suitable perforated side-vented coating pan with baffles fitted with single or multiple spray guns to produce a spray fan to cover the entire width of the tablet bed;
(11) the tablet bed was warmed in the rotating coating pan until an exhaust temperature of 40-44° C. was reached at an inlet air flow of about 270-350 CFM;
(12) the average weight of warmed uncoated tablet weight was determined as the initial weight;
(13) the sitagliptin phosphate coating suspension was sprayed onto the tablet bed at a suitable spray rate and atomization pressure;
(14) spraying with the sitagliptin phosphate coating suspension was continued while monitoring the tablet weight until the required weight gain was obtained;
(15) an approximate dried coat weight of 129 mg equivalent to 50 mg sitagliptin (as free base) or 258 mg equivalent to 100 mg of sitagliptin (as free base) was deposited over the tablet cores;
(16) spraying was stopped, and the tablets were dried and discharged from the coating pan;
(17) the Opadry color suspension was prepared by dispersing Opadry I powder in the required amount of purified water to obtain a concentration of approximately 10% (w/w);
(18) the coated tablets from step 16 were loaded into a suitable perforated side-vented coating pan with baffles fitted with single or multiple spray guns to produce a spray fan to cover the entire width of the tablet bed;
(19) the tablet bed was warmed in the rotating coating pan until an exhaust temperature of 40-44° C. was reached at an inlet air flow of about 270-350 CFM;
(20) the average weight of warmed tablet was determined as the initial starting weight;
(21) the Opadry color suspension was sprayed onto the tablet bed at a suitable spray rate and atomization pressure;
(22) spraying with the Opadry coating suspension was continued while monitoring the tablet weight until the required weight gain was obtained;
(23) an approximate dried overcoat weight of 31-33 mg equivalent was deposited over the tablet cores to produce the desired final tablet color and image;
(24) spraying was stopped, and the tablets were dried and discharged from the coating pan.

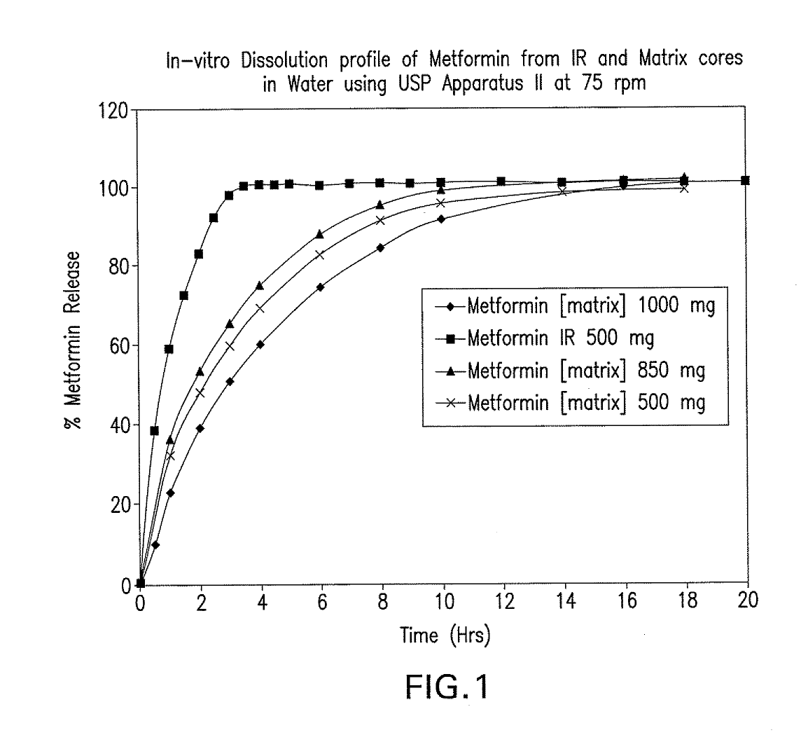

The in vitro dissolution profiles (drug release rates) for several metformin matrix tablets of the present invention were measured and are shown in FIG. 1. The three extended-release formulations produced well-differentiated metformin drug release rates with about 80% or higher of label claim being dissolved in about 4-8 hours. The duration of drug release targeted was due to a relatively narrow absorption window for metformin from the gastrointestinal tract. There is minimal absorption of metformin in the lower part of the ileum and colon, resulting in non-absorption of drug remaining in the dosage form after about 8 hours passage through the gastrointestinal tract.

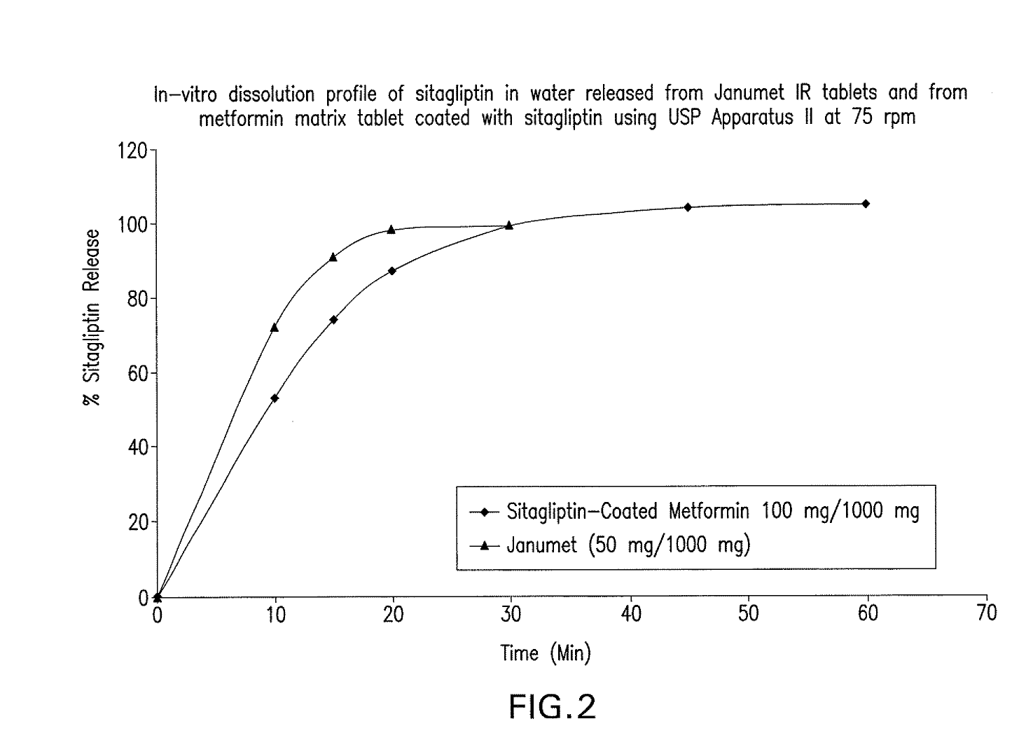

Dissolution profile of sitagliptin phosphate from the drug film layer was also measured and is shown in FIG. 2. The dissolution was found to be complete within 30 minutes and to be comparable to that of sitagliptin phosphate in JANUMET™ which is a marketed fixed-dose combination of immediate-release metformin hydrochloride and immediate-release sitagliptin phosphate.

While the invention has been described and illustrated in reference to specific embodiments thereof, those skilled in the art will appreciate that various changes, modifications, and substitutions can be made therein without departing from the spirit and scope of the invention. For example, effective dosages other than the preferred doses as set forth hereinabove may be applicable as a consequence of variations in the responsiveness of the human being treated for a particular condition. It is intended therefore that the invention be limited only by the scope of the claims which follow and that such claims be interpreted as broadly as is reasonable.

What is claimed is:

1. A pharmaceutical composition in the form of a tablet comprising an inner core matrix composition comprising metformin hydrochloride and an extended-release excipient; and further comprising an outer coat immediate-release composition comprising sitagliptin, or a pharmaceutically acceptable salt thereof, and at least one excipient.

2. The pharmaceutical composition of claim 1 wherein said extended-release excipient is a hydroxypropylmethylcellulose with an apparent viscosity of at least 10,000 cP when present in a 2% solution in water.

3. The pharmaceutical composition of claim 2 wherein said hydroxypropylmethylcellulose has an apparent viscosity of 80,000 cP when present in a 2% solution in water.

4. The pharmaceutical composition of claim 3 wherein said hydroxypropylmethylcellulose has an apparent viscosity of about 80,000 to about 120,000 cP when present in a 2% solution in water.

5. The pharmaceutical composition of claim 1 wherein said metformin hydrochloride is present in said inner core matrix composition in an amount of about 50% to about 70%.

6. The pharmaceutical composition of claim 1 wherein said inner core matrix composition further comprises a binding agent.

7. The pharmaceutical composition of claim 6 wherein said binding agent is polyvinylpyrrolidone.

8. The pharmaceutical composition of claim 6 additionally comprising one or two excipients selected from the group consisting of a glidant and a lubricant.

9. The pharmaceutical composition of claim 8 wherein said glidant is colloidal silicon dioxide and said lubricant is sodium stearyl fumarate.

10. The pharmaceutical composition of claim 1 wherein said outer coat immediate-release composition further comprises a film-forming polymer and one or more excipients selected from the group consisting of a plasticizer, a dispersing agent, a colorant, and an antioxidant.

11. The pharmaceutical composition of claim 10 wherein said film-forming polymer is HPMC 2910.

12. The pharmaceutical composition of claim 10 wherein said plasticizer is polyethyleneglycol 3350, said dispersing agent is hydrated aluminum silicate, and said antioxidant is propyl gallate.

13. The pharmaceutical composition of claim 1 wherein said pharmaceutically acceptable salt of sitagliptin is the dihydrogenphosphate salt.

14. The pharmaceutical composition of claim 1 wherein said sitagliptin is present in a unit dosage strength of 25, 50 or 100 milligrams, and said metformin hydrochloride is present in a unit dosage strength of 500, 750, 850, or 1000 milligrams.

15. A method of treating Type 2 diabetes in a human in need thereof comprising the oral administration to said human a pharmaceutical composition of claim 1.

16. The pharmaceutical composition of claim 1 further comprising a final immediate-release film coat.
